(19) United States
(12) Patent Application Publication
(10) Pub. No.: US 2009/0082854 A1
(43) Pub. Date: Mar. 26, 2009

(54) Pitted metallic implants and method of manufacturing thereof

(76) Inventor: David Cherkes, Tibernas (IL)

Correspondence Address: ROBERT G. LEV, 4766 MICHIGAN BLVD., YOUNGSTOWN, OH, 44505, US

(21) Appl. No.: 11/903,891

(22) Filed: Sep. 25, 2007

(51) Int. Cl. A61F 2/06; B23H 3/00

(52) U.S. Cl. 623/1.42; 205/640

(57) ABSTRACT

A method of fabricating a conductive prosthetic, such as a stent, with a dimpled surface comprising the steps of: (i) providing a blank; (ii) electrochemically eroding superfluous material to leave a structural skeleton, (iii) electropolishing, (iv) dimpling the surface of the structural skeleton by selectively electrochemically eroding recesses on the surface of the prosthetic and (v) impregnating the recesses with a bioactive material, and prosthetics such as stents with dimpled surfaces fabricated thereby.

### FIELD OF THE INVENTION

The present invention is directed to Metallic implants in general, and to stents in particular, having pits in the surface thereof, particularly but not exclusively to serve as a reservoir for bioactive materials, such as pharmaceutical compositions and the like.

### BACKGROUND OF THE INVENTION

There are a number of metals and alloys that are considered as being biocompatible and are used for the fabrication of implants that are used within the body and expected to function for long periods of time.

In many applications it is beneficial for such implants to release bioactive materials to aid recovery of surrounding tissue or to reduce the likelihood of trauma and infection in the vicinity of the implant.

One type of prosthetic is the stent. Stents are metal frameworks that are inserted into lumens of vessels and ducts such as arteries, veins, the intestinal tract, etc. and serve as vascular scaffolding. For example, cardiovascular stents are typically small metal coils or mesh tubes that are generally inserted during angioplastic surgery and permanently left in the artery to keep the inner wall thereof open.

Stents are required to be flexible and tough and are manufactured within narrow tolerances.

U.S. Pat. No. 5,725,548 describes a stent with a welded seam. Such welded seams are potential failure regions and reduce the reliability of such stents.

U.S. Pat. Nos. 4,733,665, US 4,776,337, US 5,421,955 and US 5,514,154 describe stents manufactured by laser cutting from tubular blank. It will be appreciated that lasers cut thorough the metal blank by locally heating to very high temperatures. This has an embrittling effect on the microstructure of the stent. Furthermore, the laser treatment tends to result in sharp edges. These edges may be smoothed by electropolishing, but at best, this results in an additional processing step, which increases production costs and increases the risk of something going wrong, causing the component to be scrapped.

Thus welding, laser cutting and other high temperature processing technologies are inherently undesirable. One fabrication route that avoids high temperature processing completely and allows room temperature fabrication is controlled electrochemical erosion. U.S. Pat. No. 6,663,765 to Cherkes, the inventor of the current invention described hereinbelow, titled “method and device for the manufacture of the medical expanding stents” discloses an electrochemical erosion process that is fast and efficient, and may be operated at room temperature with a biocompatible electrolyte such as sodium chlorate (NaClO₃), for example. This patent is incorporated herein by reference.

Invasive surgery risks infection and the site where metal is implanted into the body is prone to becoming infected. In some applications, blood clotting is desirable while in others, blood clotting is to be deterred. In general, there is a need for bioactive ingredients to be associated with bio-inert metal implants to provide desired biological effects over time and to inhibit non-desired effects. The biological effect may be the controlled release of a blood clotting agent, a blood thinning agent, an antibiotic, a fungicide and the like.

Drug-eluting stents combine the mechanical action of a stent with a local drug delivery mechanism aimed at re-enforcing the stent effect and improving the clinical outcome of the procedure. Drug eluting stents have the structural features of conventional stents, but additionally comprise an active composition, typically in the form of a polymer coating which provides a consistent and even dispensing of the drug from the stent over time.

Since 2003, drug-eluting stents have developed very fast. Rapamycin and Paclitaxel are sometimes deposited on the surface of cardiovascular stents to prevent restenosis, reclosure or re-occlusion. By way of example, Boston Scientific's Taxus™ stent is coated with a Paclitaxel drug. Johnson and Johnson also manufacture a coated stent, which is marketed as the Cypher stent.

Stents are not only useful in cardiovascular applications. Stents for treating pulmonary diseases causing the narrowing or collapsing airways such as chronic bronchitis, emphysema and asthma have also been developed. For example, Boston Scientific supplies pulmonary stents. These tube-like structures are usually placed in the trachea or primary bronchi and are designed to prop open the airway and keep it from becoming obstructed and may be used to treat advanced-stage cancerous tumors blocking the trachea or bronchi (the branches of the trachea that lead to the lungs) and non-cancerous blockages that cannot be treated by any other methods. Some of the metal stents in use are partially covered in polyurethane, which is designed to help prevent tissue from growing through the stent.

Drug eluting pulmonary stents are also known. For example, Broncus Inc. has developed a drug-eluting stent that is used in a treatment system for emphysema, and is marketed under the name Exhale™ The Exhale system seeks to take advantage of collateral ventilation typical of advanced emphysema, by creating passageways from the lung parenchyma to large airways, allowing spent air to escape the lungs during exhalation therethrough. Broncus' Exhale Drug-Eluting Stent is coated with the Paclitaxel drug, which acts again chronic inflammation.

The deposition and controlled release of active ingredients from the surface of metallic implants is not easy. Polymer coatings (both degradable and non-degradable) have been employed to bond bioactive materials to metallic implants. Polylactic acid, polyglycolic acid and polymethyl methacrylate (PMMA, commonly known as bone cement) have been used in drug eluting stents for this purpose.

There is a problem with polymer coatings however, in that polymers are not fully biocompatible, and tend to degrade over time. Since they are organic, they can serve as feedstocks for fungi and bacteria and thus are easily infected. Even poly-methyl-methacrylate, though having a long history of use in surgery from the pioneering work by Chandler, is actually carcinogenic, with the monomer being highly toxic.

US 2006/0115512 to Peacock et al. titled “Metallic structures incorporating bioactive materials and methods for creating the same” describes methods to create medical devices and implantable medical devices with an electrochemically engineered porous surface that contains one or more bioactive materials to form bioactive composite structures. The bioactive composite structures are prepared using electrochemical codeposition methods to create metallic layers with pores that can be loaded with bioactive materials. Particularly, the application relates to stents with bioactive composite structure coatings.

In codeposition of a porous surface, it will be appreciated that the stent is first manufactured and then a porous material is deposited thereonto. The active ingredient is deposited into the pores. Since faults are accumulative, it will be appreciated that the more complex the manufacturing route chosen, the more likely that something will go wrong and the component will be rejected.

Laser processing has been used for making holes in the surface of stents. Lasers tend to make through-holes, which require stopping by applying a coating of polymer thereunder. The polymer inner coating has the problems described above for polymer materials in implants. Furthermore, the perforating of the stent by through holes significantly weakens the structure of the stent, particularly because of the high temperatures generated during the laser processing. Wider stent strips are required and this adversely affects the flexibility thereof.

U.S. Pat. No. 7,055,237 to Thomas titled “Method of forming a drug eluting stent” describes a method of forming a drug eluting stent by coupling a stent framework to a mandrel, inserting into a die set having a forming surface with raised indention forming portions and closing the die set against the framework to form indentions or recesses into which drug is inserted.

Although addressing many of the problems discussed hereinabove, the solution is not really practicable, in that each stent is processed individually and is first electropolished to remove the effects of the laser or other processing and is then threaded onto a mandrel. then a die with embossed recesses is pressed into the surface thereof to produce a dimpled surface. The high pressure plastic deformation causes work-hardening and is embrittling. Furthermore, as mentioned hereinabove, additional processing stages using different equipment not only makes the manufacturing costly and time consuming, but adversely affects reliability and makes the resultant stent more prone to failure.

There remains a need for effective processing routes for fabricating metal prosthetics with pitted or dimpled surfaces for drug impregnation. Such processing routes should be low temperature, low pressure, simple manufacturing routes with minimal processing steps for fabricating the prosthetics with dimpled surfaces, so that they are substantially free of brittle recrystallization zones and residual stresses. The present invention addresses these needs.

### SUMMARY OF THE INVENTION

It is an aim of preferred embodiments of the invention to provide conductive prosthetics with pitted or dimpled surfaces for drug impregnation.

It is a further aim of the invention to provide a low temperature, low pressure, simple manufacturing route with minimal processing steps for fabricating prosthetics with dimpled surfaces that are substantially free of brittle recrystallization zones and residual stresses.

It is a specific aim of preferred embodiments to provide dimpled stents and methods of manufacturing thereof, that allow stents to be fabricated with recesses or pits in the surfaces thereof for drug elution purposes, wherein such stents retain the toughness and flexibility of the non-dimpled stents.

A first aspect of the invention is directed to a method of fabricating a conductive prosthetic with a dimpled surface comprising selective electrochemical erosion of recesses on the surface of the prosthetic.

Optionally, the prosthetic is a stent.

Optionally the stent is selected from the list of pulmonary stents and cardiovascular stents.

A second aspect of the invention is directed to a conductive prosthetic with a dimpled surface fabricated by the method of selective electrochemical erosion.

Typically the conductive prosthetic is fabricated from at least one of the list comprising a metal, an alloy, graphite, a metallically conductive composite material and a conductive polymer.

Optionally the prosthetic further comprises a bioactive material deposited in the recesses.

Optionally, the bioactive material comprises an active ingredient selected from the list of a blood clotting agent, a blood thinning agent, an antibiotic, a fungicide and a chemotherapeutic composition.

Optionally, the bioactive material comprises an active ingredient selected from the list of Rapamycin and Paclitaxel.

Typically, the conductive prosthetic is a stent.

Optionally, the stent is a pulmonary stent and the bioactive ingredient comprises at least one material selected from the list comprising an expectorant, an anti-inflammant, an anti-allergen and a bronchodilatant.

A third aspect of the invention is directed to using a prosthetic with a dimpled surface fabricated by the method of selective electrochemical erosion in a surgical procedure.

A fourth aspect of the invention is directed to a method of fabricating a metallic prosthetic with a dimpled surface comprising the steps of: (i) providing a blank; (ii) electrochemically eroding superfluous material to leave a structural skeleton, and (iv) dimpling the surface of the structural skeleton by selectively electrochemically eroding recesses on the surface of the prosthetic.

Optionally and typically, the method further comprises the step (iii) of electropolishing.

Optionally and typically, the method further comprises the step (v) of impregnating the recesses with a bioactive material.

Optionally the bioactive material is selected from the list of Rapamycin and Paclitaxel.

Optionally the bioactive material comprises at least one of the list comprising a blood clotting agent, a blood thinning agent, an antibiotic, a fungicide and a chemotherapeutic composition.

A fifth aspect of the invention is directed to a conductive prosthetic with a dimpled surface fabricated by the method comprising the steps of: (i) providing a blank; (ii) electrochemically eroding superfluous material to leave a structural skeleton, and (iv) dimpling the surface of the structural skeleton by selectively electrochemically eroding recesses on the surface of the prosthetic.

Typically the conductive prosthetic is metallic. Optionally it is fabricated from at least one of the list comprising a metal, an alloy, graphite, a metallically conductive composite material and a conductive polymer.

Typically, the conductive prosthetic is a stent.

Optionally, the conductive prosthetic is a pulmonary stent and the bioactive ingredient comprising at least one material selected from the list comprising an expectorant, an anti-inflammant, an anti-allergen and a bronchodilatant.

A sixth aspect of the invention is directed to using a dimpled prosthetic as above, in a surgical procedure.

A seventh aspect of the invention is directed to fabricating recesses on the surface of a prosthetic by room temperature electrochemical erosion.

An eighth aspect of the invention is directed to a cathode for use in electrochemical erosion of recesses on the surface of a metallic component, the cathode comprising a section with a conductive micro-pattern of recesses in relief, surrounded by an insulative layer.

Optionally, the cathode is curved. Alternatively, the cathode is flat.

Typically the metallic component is conductive tube.

Optionally the cathode further comprises a first section for fabricating a mesh from the tube by sacrificial erosion.

Optionally the cathode further comprises a smooth conductive section for electropolishing the mesh.

Optionally the cathode further comprises a subsequent section comprising conductive strips surrounded by insulative material for sectioning the metallic component.

### BRIEF DESCRIPTION OF THE FIGURES

For a better understanding of the invention and to show how it may be carried into effect, reference will now be made, purely by way of example, to the accompanying Figures, wherewith it is stressed that the particulars shown are by way of example and for purposes of illustrative discussion of the preferred embodiments of the present invention only, and are presented in the cause of providing what is believed to be the most useful and readily understood description of the principles and conceptual aspects of the invention.

FIG. 1 is an isometric projection of an exemplary metal implant, specifically a stent, in accordance with one embodiment of the invention, having a dimpled surface for accepting a bioactive material, such as a drug in the recesses thereof and showing the tubular blank from which it is constructed, about the ends thereof;

FIG. 2 shows the stent of FIG. 1, after removal of the tubular blank ends;

FIG. 3 is a schematic cross-section of a fabrication rig having a convex cathode for fabrication of the stent of FIG. 1 by room temperature controlled electrochemical erosion;

FIG. 4a is a schematic plan view of the surface of the cathode of FIG. 3;

FIG. 4b is a schematic cross-section through the cathode along A-A showing the construction thereof across the dimpled network part of the stent;

FIG. 4c is schematic cross-section through the cathode along B-B showing the construction thereof at the ends;

FIG. 5 is an isometric projection of the curved cathode of FIG. 3;

FIG. 6 is a schematic projection of a flat cathode in accordance with a second embodiment thereof, and

FIG. 7 is a flowchart illustrating a method of fabricating prosthetic implants and other components with dimpled surfaces.

### DESCRIPTION OF PREFERRED EMBODIMENTS

The present invention is directed to providing conductive components with micro-dimpled surfaces that are suitable for implantation within the human body, such as stents for example, and to methods of fabricating such components.

The conductive components are typically metallic and may comprise a metal, an alloy, graphite, a metallically conductive composite material and a conductive polymer.

The micro-dimpled surface of the components includes a number of recesses that serve as micro-reservoirs for bioactive materials, such as polymeric matrices impregnated with pharmaceutical compositions such as blood clotting agents, blood thinning agents, antibiotics, fungicides and/or chemotherapeutic compositions.

The recesses of micro-dimpled components of embodiments the invention may serve as reservoirs for drugs. Specific pharmaceuticals that have been successfully used in drug eluting cardiovascular stents include Rapamycin or Paclitaxel and the micro-dimpled surface of metallic components fabricated in accordance with the invention may be impregnated with these drugs or with other bioactive materials.

It is a specific feature of the present invention that the recesses on the prosthetic are fabricated by a sacrificial electrochemical erosion technique wherein superfluous material is etched away. This is in contradistinction to the electrodeposition techniques described in US 2006/0115512 to Peacock et al. The sacrificial electrochemical erosion technique used for fabricating the recesses is a variation of the processing described in U.S. Pat. No. 6,663,765 to Cherkes. Indeed, a preferred manufacturing route for stents is to fabricate the skeletal structure thereof and to then dimple the surface of the skeletal structure with a pattern of recesses, all by electrochemical erosion in a continuous manufacturing process that may include polishing and cutting to size.

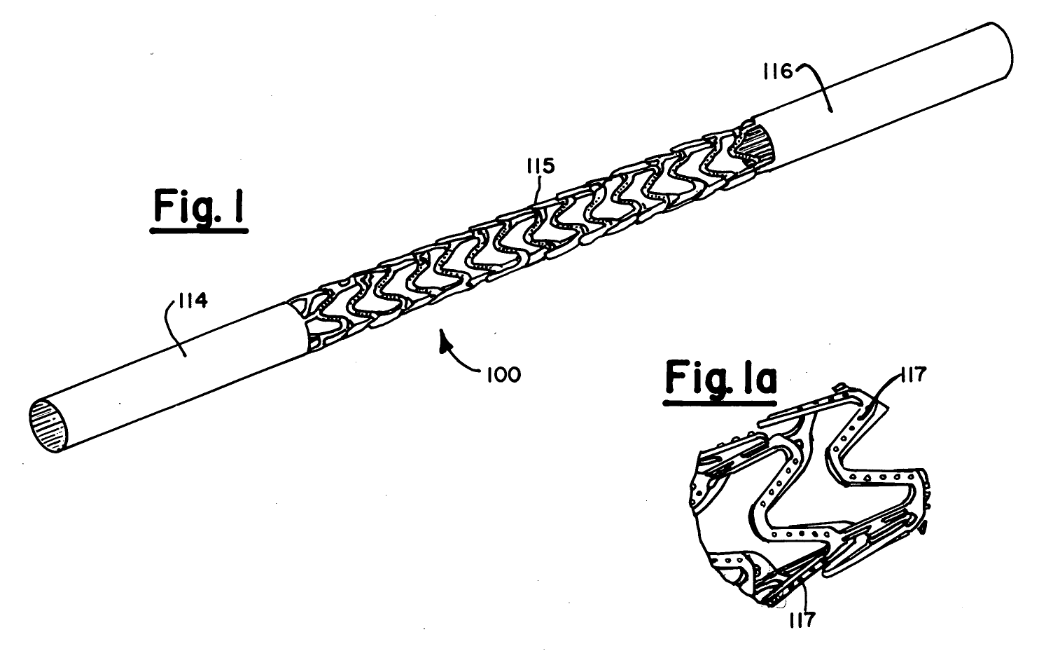

With reference to FIG. 1, an isometric projection of a stent precursor 100 in accordance with one embodiment of the invention is shown. Stent precursor 100 has smooth cylindrical ends 114, 116, and a central part 115 that is an expandable cylindrical mesh having an open network structure. It is a particular feature of implants in accordance with preferred embodiments of the invention, that the surface thereof is dimpled, with strategically placed recesses 117 thereupon for accepting a bioactive material, such as a drug, for example.

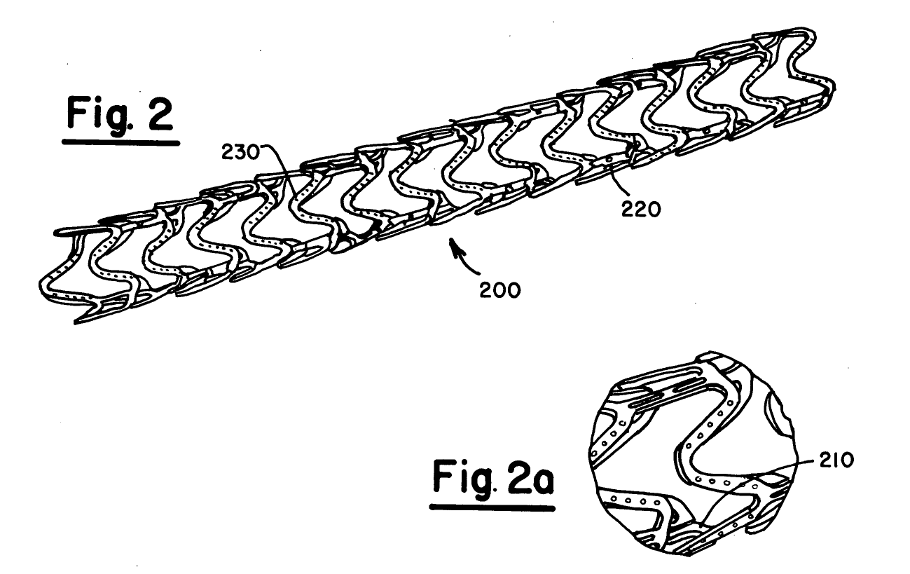

As shown in FIG. 2, an enlarged view of a stent 200 cut from the central part 115 of the Stent precursor 100 (FIG. 1) showing the structure thereof is shown. Stent 200 comprises expandable crenellated rings 210 joined by connecting elements 220, and can be dilated, perhaps by a balloon inserted thereinto, to serve as a scaffolding structure to support a blood vessel, for example. As will be explained hereinbelow, the stent 200 may be fabricated by electrochemical erosion of from a tubular cursor using a principle disclosed in U.S. Pat. No. 6,663,765.

Unlike the stents described in U.S. Pat. No. 6,663,765 however, it is a particular feature of stents in accordance with embodiments of the invention, that the surfaces thereof are micro-dimpled with pores 230 that provide anchoring points for tissue growth thereinto, and may be filled with a bioactive material such as a pharmaceutical composition for example.

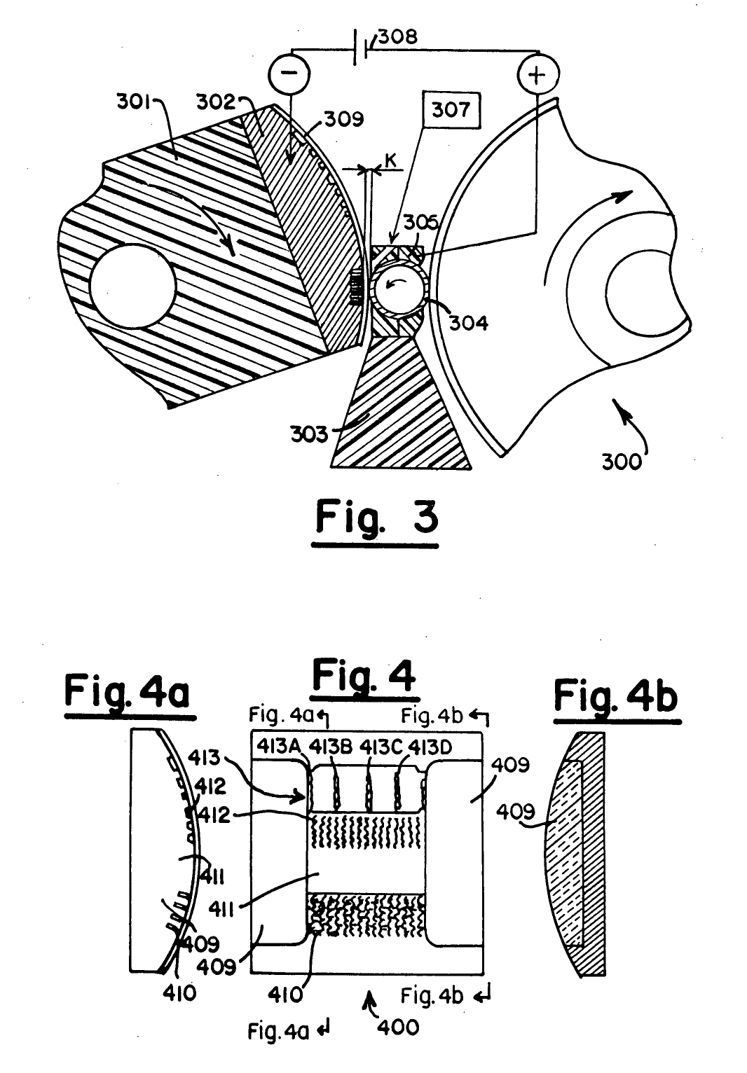

With reference to FIG. 3, a schematic cross-section of a fabrication rig 300 for room temperature controlled electrochemical erosion fabrication of the stent of FIG. 1 is shown, having a convex cathode 302 mounted on a rotor 301 that electrochemically erodes material from a tubular blank 304 mounted on a mandrel 305 supported by a frame 303. The tubular blank 304 is rolled across the surface of the cathode 302 by an auxiliary roller 306. Electrical power source 308 maintains a potential difference between the mandrel 305 and the cathode 302; the mandrel 305 being maintained at a higher potential than the cathode 302. A pump 307 pumps electrolyte over the contact surface between the tubular blank 304 and the cathode 302 and metal ions are chemically etched away from the tubular blank 304, by metals on the tubular blank 304 being ionized into solution by oxidization, by having electrons stripped therefrom, towards the cathode 302.

As explained in U.S. Pat. No. 6,663,765, the mandrel 305 includes an electrically non-conducting casing which can be PTFE, for example. A metal bushing is positioned within the casing, which extends a distance of about 0.1 to about 0.2 mm beyond the metal bushing to prevent contact between the metallic bushing and the electrolyte.

The stent blank 304 is inserted into a hole formed in the metal bushing and is supported by a pair of metal rests which supply electric power to the stent blank 304 from the positive pole of the power source 308 through one or more screws into the bushing. In this manner, electrical current is supplied to both sides of the tubular stent blank 304 simultaneously.

The distance separating the surface of the cathode 302 and the tubular stent blank 304 depends on the current strength and the specific electrolyte used for stent processing, which may be optimized for different stent materials and dimensions, and is typically about 0.05 mm. Both the rotator 301 and the stent blank 304 are simultaneously rotated by drive roller 306 which rotates the blank 304 with respect to the cathode plate 302 without slippage. During processing, the rotator 301 with its attached cathode plate 302 and the tubular blank 304, are rotated simultaneously in the direction shown by arrows, such that the stent blank 304 makes multiple revolutions. An electrolyte is supplied under pressure between the stent blank 304 and cathode plate 302. The electrolyte is system specific and depends, inter alia, on the material from which the stent blank 304 is fabricated. During the first revolution the stent pattern shown in FIG. 1 is electrochemically formed by rolling the stent blank 304 over the patterning section 410. During the second revolution, the resulting mesh is electropolished by being rolled over smooth polishing section 411 and then cut by cutting section 413, which includes a number of cutting strips 413A-E, that, as described in U.S. Pat. No. 6,663,765, may preferably be individually activated to allow the stent blank 304 to be cut into desired length.

In contradistinction to the prior art described in U.S. Pat. No. 6,663,765, prior to cutting the mesh 115 (FIG. 1) created from the tubular blank 304 (FIG. 3) into individual stents 200 (FIG. 2) by conductive strips 413A-E in section 413, a dimpling process 412 is applied to create recesses 230 (FIG. 2), i.e. a pattern of pits or recesses in the surface of the mesh 115. These recesses 230 may be impregnated by a pharmaceutical composition in a polymeric matrix, for combating inflammation or infection, for example, but other bioactive materials, such as calcium hydroxyapatite or collagen for promoting tissue growth may be subsequently deposited in the recesses.

The ends 409 of the cathode 400 is preferably rough and insulative and helps roll the tubular blank 304 over the surface of the cathode 400.

It will be appreciated that there is tremendous flexibility in the shape size and depth of the recesses 230. Typically the recesses are not through-holes but are rather pit like. They may be through-holes however. The characteristics of the pits are a function, inter alia of processing time, cathode surface, applied current, material and thickness of the blank. It is possible to manufacture recesses with sub micron dimensions.

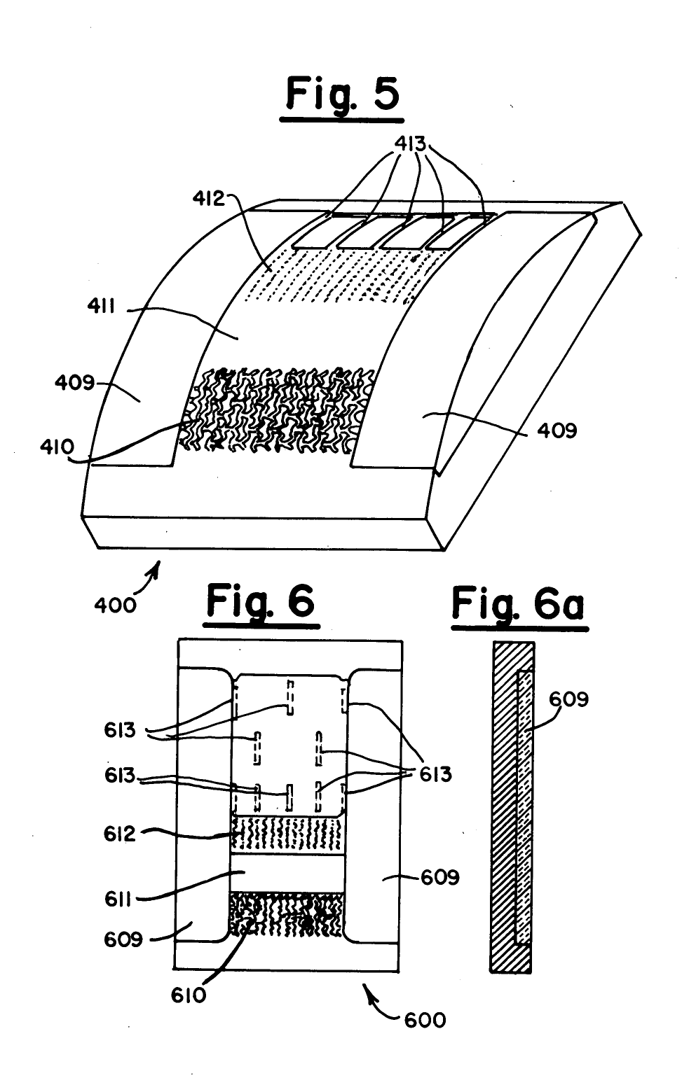

With reference to FIGS. 4a and 4b, showing a schematic plan view of the surface of the cathode of FIG. 3 and a schematic cross-section there across along A-A, and FIG. 5 showing the cathode 400 in isometric perspective; an open mesh section 115 (FIG. 1) may be fabricated, polished and cut from a tubular blank 304, by a curved cathode plate 4 having a mesh fabrication section 410, an electropolishing section 411 and an electro-sectioning section 413. The mesh fabrication section 410 is a metallic pattern that is identical to that to be cut from the tubular blank 304.

The conductive mesh pattern 410 is fabricated onto the curved cathode plate 400 which may be fabricated from a range of conductive materials, such as gold, platinum, or alloys of same, stainless steel, brass or other copper alloys or from graphite, for example. The outline is created using one or more of etching, drilling, photochemical, electroerosion or other process steps known in the prior art to remove material from surface of cathode 302 to a depth of up to about 0.2 mm to leave the conductive mesh pattern 410 in relief. The remaining lowered portions of the cathode plate 400 are filled with an insulating material, such as a self-hardening polymeric resin, for example.

The basic processing method for fabricating stents by controlled electrochemical erosion is disclosed in U.S. Pat. No. 6,663,765, mutatis mutandis. However, unlike the method of U.S. Pat. No. 6,663,765, the cathode 400 of the present invention also includes a dimpling section 412 for applying recesses to the open mesh resulting from the electrochemical erosion by mesh forming section 410. Dimpled section 412 consists of arrays of isolated metallic contact points in an insulating polymer surface. Rolling the mesh network resulting from the first stage over the dimpling section 412 in the presence of an electrolyte and an electric current, causes recesses to be electrochemically etched onto the surface of the mesh network.

Cutting section 413 allows the dimpled mesh of the stent precursor 100 to be cut into individual stents 200. The cutting surfaces 413A-E are metal edges that protrude in relief from the insulative surround. Preferably, as described in U.S. Pat. No. 6,663,765, the cutting surfaces 413A-E may be individually activated by supplying current thereto, so that they can be activated individually or in any combination to electrochemically cut stents in a range of desirable sizes.

With further reference to FIG. 4c, which is schematic cross-section through the cathode 400 along B-B, the ends of the cathode are typically insulative and rough to provide good traction for rolling the tubular blanks 304 therealong.

Although for reasons elaborated on in U.S. Pat. No. 6,663,765, that are readily apparent from FIG. 3, the cathode 400 is often curved, it will be appreciated that it may be flat, and FIG. 6 is a schematic projection of a flat cathode 600 in accordance with a second embodiment thereof, again having rough, insulative ends 609, mesh fabrication section 610, an electropolishing section 611, a dimpling session 612 and an electro-sectioning section 613 for cutting the resultant stent precursor to size.

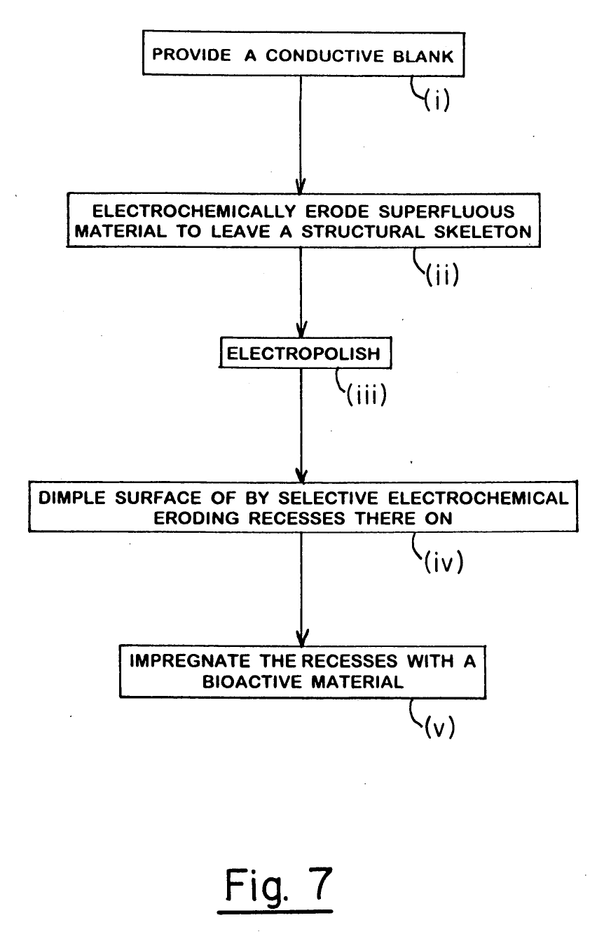

With reference to FIG. 7, one method of fabricating a metallic prosthetic, such as a stent, with a dimpled surface comprises the steps of: (i) providing a blank; (ii) electrochemically eroding superfluous material to leave a structural skeleton, (iii) electropolishing, (iv) dimpling the surface of the structural skeleton by selectively electrochemically eroding recesses on the surface of the prosthetic and (v) impregnating the recesses with a bioactive material.

The processing route described hereinabove has several advantages over laser treatment and pressure forming. Notably, the processing is at room temperature and neither pressure nor heat are applied to fabricate the recesses in the surface thereof. This avoids work hardening and recrystallization of the microstructure of the stent metal and maintains the strength and flexibility of the stent. The recesses are made without deforming the stent and are not themselves deformed by expansion of the stent. Consequently the mechanical properties of the stent, in particular the flexibility thereof, are not compromised.

Since the recesses are fabricated in one operation with the mesh itself and the polishing thereof, the fabrication process is relatively simple and easily automated. The fabrication process is easy to monitor and inherently reliable, giving high yields in a cost effective manner.

The designer has a great deal of flexibility in stent design and in the position, number, shape and size of the recesses.

A polymer impregnated with a bioactive material may be used for filling the recesses, from which the bioactive/pharmaceutical ingredient may then be eluted. The polymer may include poly-α-hydroxy acid esters such as polylactic acid, polyglycolic acid, polylactic-co-glycolic acid, polylactic acid-co-caprolactone; polyethylene glycol and polyethelene oxide, polyvinyl pyrrolidone, polyorthoesters. Additionally or alternatively, it may include polysaccharides and polysaccharide derivatives such as polyhyaluronic acid, polyalginic acid, chitin, chitosan, cellulose, hydroxyehtylcellulose, hydroxypropylcellulose and carboxymethylcellulose. It may include polypeptides and/or proteins such as polylysine, polyglutamic acid, albumin; polyanhydrides; polyhydroxy alkonoates such as polyhydroxy valerate, polyhydroxy butyrate, and the like. Indeed, any polymer that is compatible with the active ingredient and the metal matrix may be used.

The therapeutic or bioactive agents used in the present invention may include classical low molecular weight drugs including all classes of action as exemplified by, but not limited to: antineoplastic, immuno-suppressants, antiproliferatives, antithrombins, antiplatelet, antilipid, anti-inflammatory, angiogenic, anti-angiogenic, vitamins, ACE inhibitors, vasoactive substances, antimitotics, metello-proteinase inhibitors, NO donors, estradiols, anti-sclerosing agents, alone or in combination. The therapeutic agent may also/alternatively include one or more biologic agents or higher molecular weight substances with drug-like effects on target tissue. These may include peptides, lipids, protein drugs, enzymes, oligonucleotides, ribozymes, genetic material, prions, virus, bacteria, and eucaryotic cells such as endothelial cells, monocyte/macrophages or vascular smooth muscle cells, for example. The recesses may be impregnated with a therapeutic agent such as a pro-drug, which metabolizes into the desired drug when administered to a host. In addition, the therapeutic agents may be pre-formulated as microcapsules, microspheres, microbubbles, liposomes, niosomes, emulsions, dispersions or the like before it is incorporated into a polymer or other carrier and deposited into the recesses. The therapeutic agent may also include radioactive isotopes or agents activated by some other form of energy such as light or ultrasonic energy, or by other circulating molecules that can be systemically administered.

Thus the scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1. A method of fabricating a conductive prosthetic with a dimpled surface comprising selective electrochemical erosion of recesses on the surface of the prosthetic.

2. The method of claim 1 wherein said prosthetic comprises a stent.

3. A conductive prosthetic with a dimpled surface fabricated by the method of claim 1.

4. The conductive prosthetic of claim 3 further comprising a bioactive material deposited in the recesses thereon.

5. The conductive prosthetic of claim 4 wherein said bioactive material comprises an active ingredient selected from the list comprising blood clotting agents, blood thinning agents, antibiotics, fungicides, chemotherapeutic compositions, expectorants, anti-inflammants, anti-allergens, bronchodilatants, Rapamycin and Paclitaxel.

6. The conductive prosthetic of claim 4 wherein said bioactive material is co-deposited with a polymeric material into the recesses.

7. The conductive prosthetic of claim 3 being a stent.

8. Use of the prosthetic of claim 3 in a surgical procedure.

9. The prosthetic of claim 3 being fabricated from at least one of the list comprising a metal, an alloy, graphite, a metallically conductive composite material and a conductive polymer.

10. A method of fabricating a conductive prosthetic with a dimpled surface comprising the steps of:
(i) providing a blank;
(ii) electrochemically eroding superfluous material to leave a structural skeleton, and
(iv) dimpling the surface of the structural skeleton by selectively electrochemically eroding recesses on the surface of the prosthetic.

11. The method of claim 10 further comprising the step (iii) of electropolishing.

12. The method of claim 10 further comprising the step (v) of impregnating the recesses with a bioactive material.

13. The method of claim 10 wherein said bioactive material is selected from the list comprising blood clotting agents, blood thinning agents, antibiotics, fungicides, chemotherapeutic compositions, Rapamycin, Paclitaxel, expectorants, anti-inflammants, anti-allergens, bronchodilatants and mixtures thereof.

14. A conductive prosthetic with a dimpled surface fabricated by the method of claim 10.

15. The metal prosthetic of claim 14 comprising a stent.

16. Use of the prosthetic of claim 14 in a surgical procedure.

17. The prosthetic of claim 14 being fabricated from at least one of the list comprising a metal, an alloy, graphite, a metallically conductive composite material and a conductive polymer.

18. A method of fabricating recesses on the surface of a prosthetic by room temperature electrochemical erosion.

19. A cathode comprising a section with a conductive micro-pattern of recesses in relief, surrounded by an insulative layer for use in electrochemical erosion of recesses on the surface of a conductive component.

20. The cathode of claim 19 being selected from the list of convex cathodes and flat cathodes.

21. The cathode of claim 19 wherein the conductive component is metallic tub.

22. The cathode of claim 19 comprising a first section for fabricating a mesh from the tube by sacrificial electrochemical erosion.

23. The cathode of claim 19 further comprising a smooth conductive section for electropolishing the mesh.

24. The cathode of claim 19 further comprising a subsequent section comprising conductive strips surrounded by an insulative material for sectioning the conductive component.

25. The cathode of claim 19 wherein said conductive micro-pattern comprises metallic, graphitic, alloy or otherwise conductive materials.
